(19) United States
(12) Patent Application Publication
(10) Pub. No.: US 2018/0365959 A1
(43) Pub. Date: Dec. 20, 2018

(54) Method for Posture and Body Position Correction

(71) Applicants: David R. Hall, Provo, UT (US); Nathan Davis, Bountiful, UT (US); Joe Fox, Spanish Fork, UT (US); William H. Reynolds, Orem, UT (US)

(72) Inventors: David R. Hall, Provo, UT (US); Nathan Davis, Bountiful, UT (US); Joe Fox, Spanish Fork, UT (US); William H. Reynolds, Orem, UT (US)

(21) Appl. No.: 15/622,660

(22) Filed: Jun. 14, 2017

(51) Int. Cl. G08B 21/04; A61B 5/103; A61B 5/11; A61B 5/00

(52) U.S. Cl. CPC G08B 21/0461; A61B 5/1036; A61B 5/1116; A61B 5/6803; A61B 5/6897; A61B 5/6804; A61B 5/0004; A61B 5/6891

(57) ABSTRACT

A method for improving body positioning are disclosed. A plurality of sensors in communication with a human body sitting or standing at a table or desk are provided. Each of the plurality of sensors is configured to detect at least one position of the human body, which is compared to a standard, and at least one signal related to the at least one position is generated. A controller receives the at least one signal from each of the plurality of sensors, stores a program that evaluates the at least one position for any improper position, and generates at least one position improvement reminder, including corrections and approbations, both selected from the group comprising visual, auditory, and physical notifications. The corrections continue during a period the improper position is detected. The approbations are provided after the period and as positive feedback after intervals of proper position.

### FIELD OF THE INVENTION

This invention relates generally to ergonomics. More particularly, we are interested in providing a system to correct posture and body positioning.

### BACKGROUND

Sitting at a desk for long hours is difficult at best for even healthy, well-trained individuals. Many solutions have been provided that remind individuals to stand and move around, to keep their wrists off the desk, to stop slouching, or other posture and body positioning defects. However, a complete solution that corrects multiple posture and body positioning issues simultaneously, with both correction and positive feedback, is needed.

United States patent application number 20110054359, to Sanonov, et al., teaches a footwear-based body weight monitor and postural allocation, physical activity classification, and energy expenditure calculator. The present disclosure differs from this prior art disclosure since the prior art disclosure is used for determining posture of a standing, walking, or running person, not a sitting person. This prior art disclosure is pertinent and may benefit from the methods disclosed herein and is hereby incorporated for reference in its entirety for all that it teaches.

United States patent application number 20090260426, to Lieberman, et al., teaches a method for determining postural stability. The present disclosure differs from this prior art disclosure in that the prior art disclosure is used to determine the posture of a person standing or walking, and not sitting. This disclosure is pertinent and may benefit from the methods disclosed herein and is hereby incorporated for reference in its entirety for all that it teaches.

U.S. Pat. No. 5,425,378, to Swezey, et al., teaches a headgear-based posture-monitoring device. The present disclosure differs from this prior art disclosure in that only a single sensor is included, allowing for control of only a single position, not a plurality of positions. This single degree of freedom is insufficient for proper posture and human positioning. For example, the head can be kept perfectly level, as this device trains, but the user can still slouch significantly, negating benefits from the single position monitoring. This prior art disclosure is pertinent and may benefit from the methods disclosed herein and is hereby incorporated for reference in its entirety for all that it teaches.

U.S. Pat. No. 6,682,351, to Abraham-Fuchs, et al., teaches a method and system for monitoring the posture of a user at a training apparatus. The present disclosure differs in that the prior art disclosure detects kinematic data, not static data. This prior art disclosure is pertinent and may benefit from the methods disclosed herein and is hereby incorporated for reference in its entirety for all that it teaches.

U.S. Pat. No. 6,673,027, to Fischer, teaches a posture measurement and feedback instrument for seated occupations. The present disclosure differs in that the prior art disclosure has only one sensor. As discussed in detail above, this single degree of freedom is insufficient for proper posture and human positioning. This prior art disclosure is pertinent and may benefit from the methods disclosed herein and is hereby incorporated for reference in its entirety for all that it teaches.

U.S. Pat. Nos. 8,083,693 and 8,932,236, to McKeon, et al., teach systems for monitoring posture. The present disclosure differs in that the prior art disclosures have only one sensor. As discussed in detail above, this single degree of freedom is insufficient for proper posture and human positioning. These prior art disclosures are pertinent and may benefit from the methods disclosed herein and are hereby incorporated for reference in their entirety for all that they teach.

International patent publication number 2016055848, to Lu, et al., teaches an apparatus and a method for detecting the posture of the anatomy of a person. The present disclosure differs in that the prior art disclosure contains a sensor or sensors that only sense the posture of the neck. Each of the sensors are used for one position correction operation. As such, even with multiple sensors, only a single posture position is corrected rather than a plurality of posture positions. This prior art disclosure is pertinent and may benefit from the methods disclosed herein and is hereby incorporated for reference in its entirety for all that it teaches.

U.S. Pat. No. 5,570,301, to Barrus, teaches a system for unencumbered measurement and reporting of body posture. The present disclosure teaches against the user being aware of the presence or activity of sensors. Specifically, the posture and body positioning of the user is not shown to the user. As such, this system cannot be used for live correction of posture defects. This prior art disclosure is pertinent and may benefit from the devices disclosed herein and is hereby incorporated for reference in its entirety for all that it teaches.

### SUMMARY

A method for improving body positioning is disclosed. A plurality of sensors in communication with a human body sitting in a chair or standing at a table or desk are provided. Each of the plurality of sensors is configured to detect at least one position of the human body, compare the at least one position to a standard, and to generate at least one signal related to the at least one position. A predetermined human body posture standard, or a user defined human body posture standard, may be visually displayed to a user as the sensors are activated. The plurality of sensors comprises a plurality of weight sensors, one or more pressure sensors, one or more position sensors, and a plurality of ultrasonic transmitters/receivers, or combinations thereof. A controller receives the at least one signal from each of the plurality of sensors. The controller stores a program that evaluates the at least one position from each of the plurality of sensors for any improper position, and, for each improper position, generates at least one position improvement reminder. The at least one position improvement reminder includes corrections and approbations, both selected from the group comprising visual, auditory, and physical notifications. The corrections continue during a period the improper position is detected. The approbations are provided after the period and as positive feedback after intervals of proper position. The physical notifications comprise vibrating portions of the chair, vibrating a keyboard wrist support on the desk, vibrating a mouse wrist support on the desk, electrically shocking a lower back portion of the human body, or combinations thereof.

The plurality of sensors may comprise a plurality of weight sensors, one or more pressure sensors, one or more position sensors, a plurality of ultrasonic transmitters/receivers, or combinations thereof. The plurality of weight sensors may be positioned in a seat of a chair such that a weight distribution of the human body sitting in the chair is evaluated.

The one or more pressure sensors may be positioned in or on a top-back portion of a chair, in or on a seat of the chair, in or on a floor, in or on a laptop such that wrists rest on the one or more pressure sensors when using an improper typing position, in or on a desk such that the wrists rest on the one or more pressure sensors when using the improper typing position, in or on a wrist support such that the wrists press on the one or more pressure sensors when using the improper typing position, on a wrist or wrists such that the wrists press the one or more pressure sensors on a desktop or laptop when using the improper typing position, or in or on a combination thereof.

The one or more position sensors may be positioned in or on a mouse, keyboard, chair, the monitors, ID badge, glasses, shoes, shirt, pants, hat, hair accessory, or a combination thereof. Position receivers may be provided that receive signals from the one or more position sensors. The signals from the position receivers may be evaluated to triangulate a location in space of the one or more position sensors. The one or more position sensors may comprise real-time locating system tags. The real-time locating system tags may comprise radio frequency communication, infrared communication, acoustic communication, or combinations thereof.

The plurality of ultrasonic transmitters/receivers may produce ultrasonic sound and detect reflections of ultrasonic sound around the human body and produce a signal for use in producing a three-dimensional map of the human body and evaluating the at least one position of the human body.

The controller may be a personal computer comprising a webcam, the webcam sensing the at least one position.

The physical reminders may comprise vibrating portions of a chair, vibrating a computer mouse, vibrating a keyboard, delivering a mild electric shock, or combinations thereof. The visual reminders may comprise on-screen text reminders, on-screen images, flashing lights, blinking lights, or combinations thereof. The auditory reminders may comprise computer beeps, audio or music reminders over computer speakers or headphones, or combinations thereof.

### BRIEF DESCRIPTION OF THE DRAWINGS

In order that the advantages of the invention will be readily understood, a more particular description of the invention briefly described above will be rendered by reference to specific embodiments illustrated in the appended drawings. Understanding that these drawings depict only typical embodiments of the invention and are not therefore to be considered limiting of its scope, the invention will be described and explained with additional specificity and detail through use of the accompanying drawings, in which:

FIG. 1 shows an office workstation.

FIGS. 2A-C show a side, front, and top view of a chair.

FIG. 3 shows an isometric view of a chair.

FIG. 4 shows an isometric view of a keyboard with wrist support.

FIG. 5 shows an isometric view of a laptop.

FIG. 6 shows an isometric view of a laptop.

FIG. 7 shows a method for improving body positioning.

### DETAILED DESCRIPTION

It will be readily understood that the components of the present invention, as generally described and illustrated in the Figures herein, could be arranged and designed in a wide variety of different configurations. Thus, the following more detailed description of the embodiments of the invention, as represented in the Figures, is not intended to limit the scope of the invention, as claimed, but is merely representative of certain examples of presently contemplated embodiments in accordance with the invention.

The term “body position” includes position of all parts of a human body. The term “posture” generally refers to the manner in which a person sits or the orientation of neck and back. In this disclosure, the term “posture” is considered a subset of “body position.” Therefore, body position includes posture, but is not limited to posture.

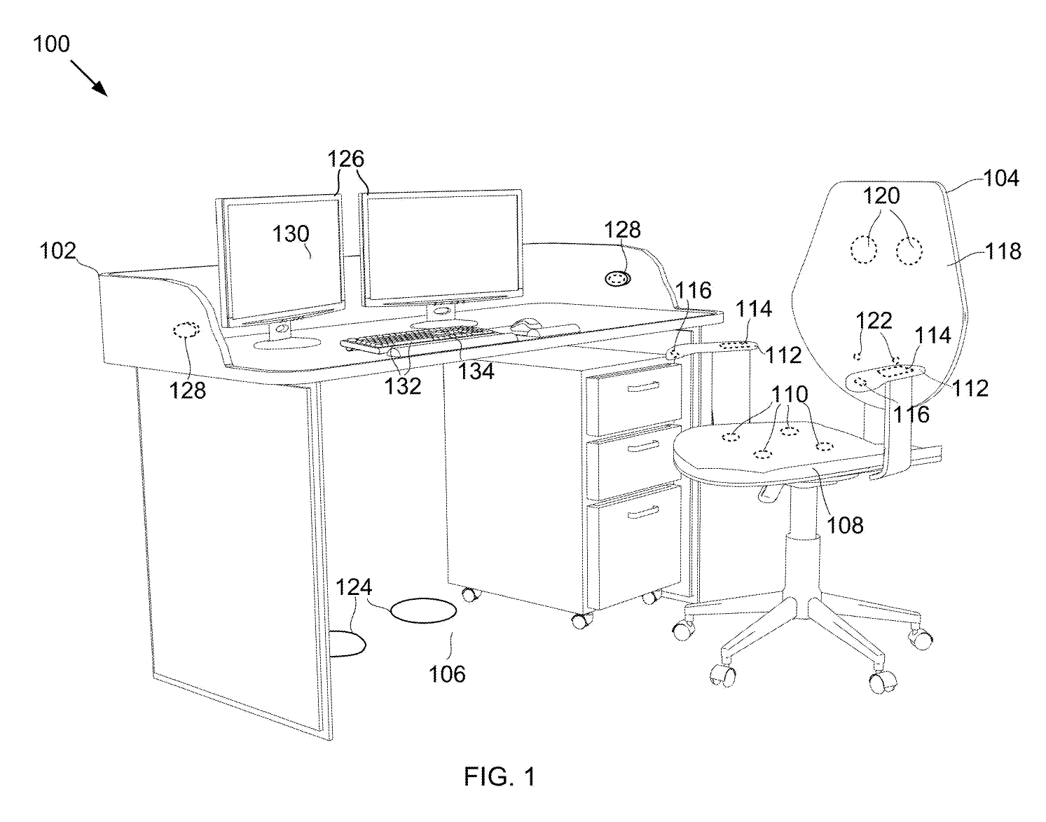

Referring to FIG. 1, an office workstation is shown at 100, as per one embodiment of the present invention. Desk 102, chair 104, and floor 106 comprise a plurality of sensors. The plurality of sensors are in communication with a human body sitting in chair 104 at desk 102 and are configured to detect at least one position of the human body, compare the at least one position to a standard, and to generate at least one signal related to the at least one position. A predetermined human body posture standard, or a user defined human body posture standard, may be visually displayed to a user as the sensors are activated. A controller, comprising a laptop or desktop computer (not shown for clarity), receives the at least one signal from each of the plurality of sensors. The controller stores a program that evaluates the at least one position from each of the plurality of sensors for any improper position, and, for each improper position, generates at least one position improvement reminder selected from the group comprising a visual reminder, an auditory reminder, and a physical reminder. Seat 108 of chair 104 comprises weight sensors 110, which detect the weight distribution of the human body sitting in chair 104. The weight sensors can be calibrated so that deviations from the desired weight distribution are noted by the controller. Arms 112 of chair 104 comprise pressure sensors 114 and position sensors 116. Pressure sensors 114 are configured to detect user arm and elbow position and force applied to arms 112 and determine deviations from proper arm and elbow position. Position sensors 116 are placed in the fronts of arms 112 and their locations in three-dimensional space are detected by position receivers (not shown). This allows the distance between the chair and the desk to be detected and deviations from proper distance to be determined. Back 118 of chair 104 comprises pressure sensors 120 and electrical probes 122. Pressure sensors 120 detect whether the user's shoulders are positioned correctly against the back of chair 104. Electrical probes 122 provide a physical reminder of improper body position by providing a mild shock to the lower back, which causes the user to abruptly correct their body position. Floor 106 comprises foot pressure sensors 124 which detect whether a user has their feet properly placed on the ground while sitting in the chair. Desk 102 comprises monitors 126, ultrasonic transmitters/receivers 128, keyboard wrist pressure sensors 132, mouse wrist pressure sensor 134. Ultrasonic transmitters/receivers 128 produce ultrasonic sound which reflects off the office workstation and the user. This ultrasonic sound is then detected by ultrasonic transmitters/receivers 128. The signals from the ultrasonic transmitters/receivers are evaluated in the controller to form a three-dimensional map of the user and determine deviations in body positioning. Keyboard wrist pressure sensors 132 detect pressure from the user's wrist when the user types with incorrect wrist position. Mouse wrist pressure sensor 134 detect pressure from the user's wrist when the user operates the mouse with incorrect wrist position. Incorrect wrist position is shown by pressure on mouse wrist pressure sensor 134 or keyboard wrist pressure sensor 132, as pressure on these sensors indicates the user is resting their wrist or wrists, rather than keeping their wrists elevated. Monitors 126 shows visual reminders 130. In some embodiments, these visual reminders are pop-up windows, flashing lights, or other typical visual reminders used by consumer electronics. In some embodiments, auditory reminders are provided through desktop speakers, laptop speakers, headphones, or audible computer beeps. The standard includes both the desired positions of the body and acceptable deviations from the desired positions. In some embodiments, the standard is determined by the program. In other embodiments, the user determines the standard. A predetermined human body posture standard, or a user defined human body posture standard, may be visually displayed to a user as the sensors are activated.

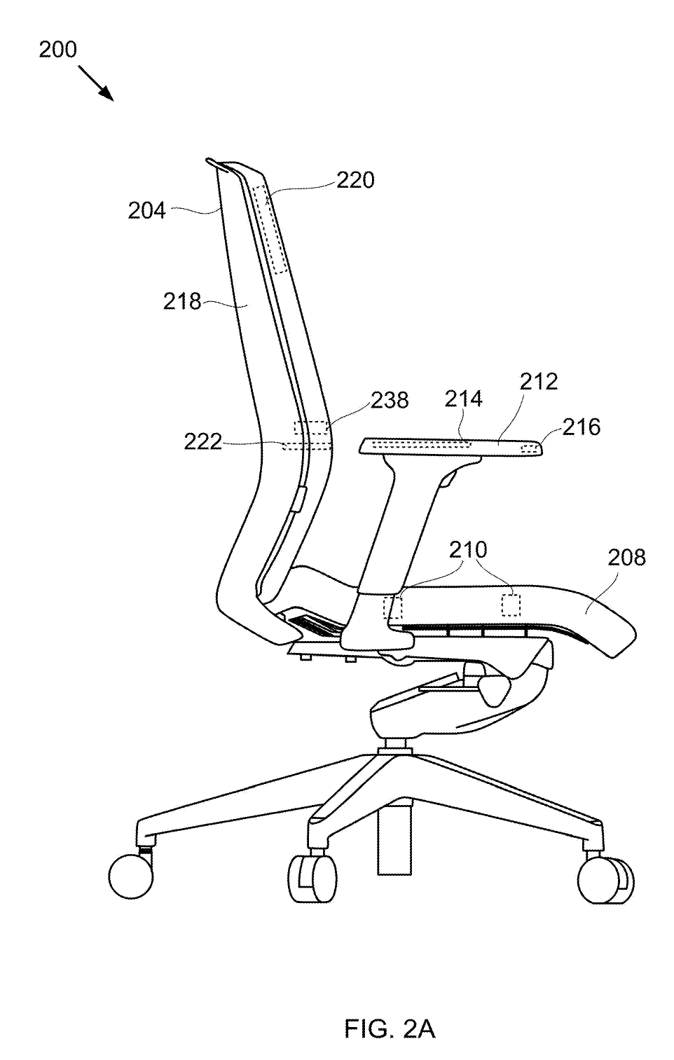

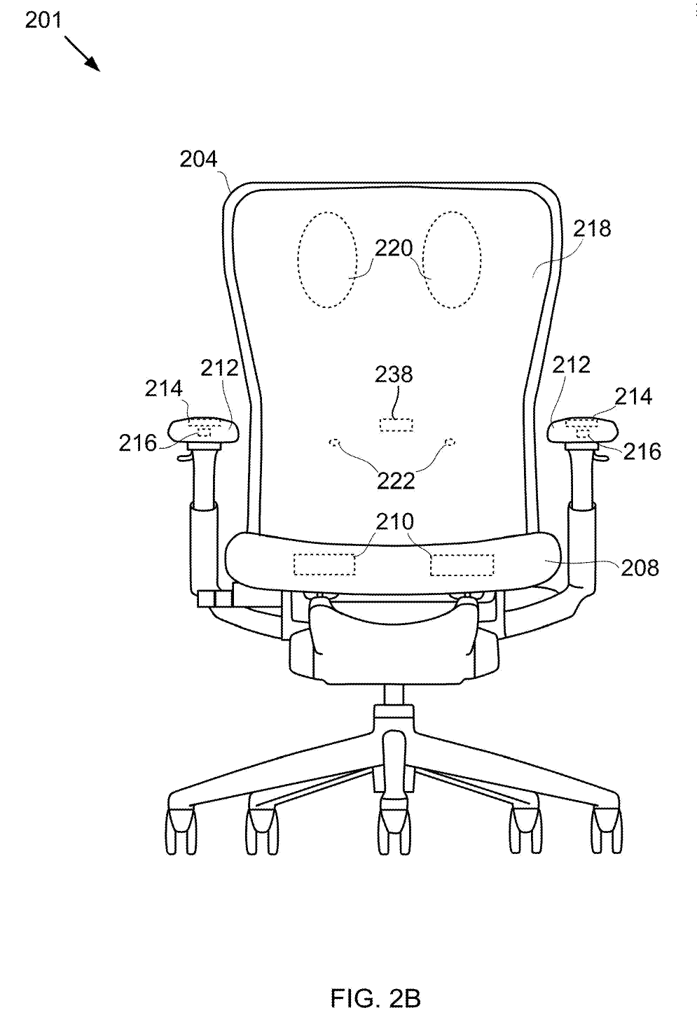

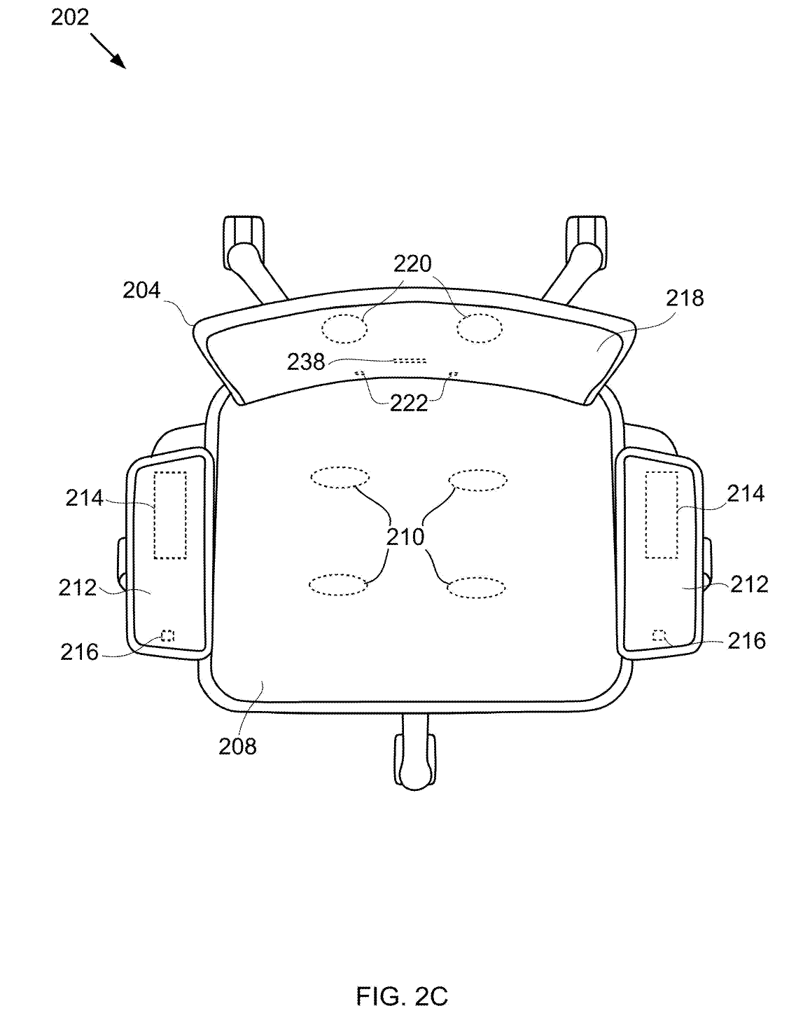

Referring to FIGS. 2A-C, a side, front, and top view of a chair are shown at 200, 201, and 202, respectively, as per one embodiment of the present invention. Chair 204 comprises sensors configured to detect at least one position of the human body and devices to produce physical reminders. This chair can be used with a controller, as in FIG. 1. Seat 208 of chair 204 comprises weight sensors 210, which detect the weight distribution of the human body sitting in chair 204. Arms 212 of chair 204 comprise pressure sensors 214 and position sensors 216. Pressure sensors 214 are configured to detect user arm and elbow position and force applied to arms 212. Position sensors 216 are placed in the fronts of arms 212 and their locations in three-dimensional space are detected by position receivers (not shown). Back 218 of chair 204 comprises pressure sensors 220, electrical probes 222, and vibratory device 238. Pressure sensors 220 detect whether the user's shoulders are positioned correctly against the back of chair 204. Electrical probes 222 provide a physical reminder of improper body positioning by providing a mild shock to the lower back, which causes the user to abruptly correct their body position. Vibratory device 238 provides a physical reminder of improper body positioning by causing back 218 of chair 204 to vibrate. In some embodiments, vibratory device 238 is used as a gentle reminder before electrical probes 222 are used.

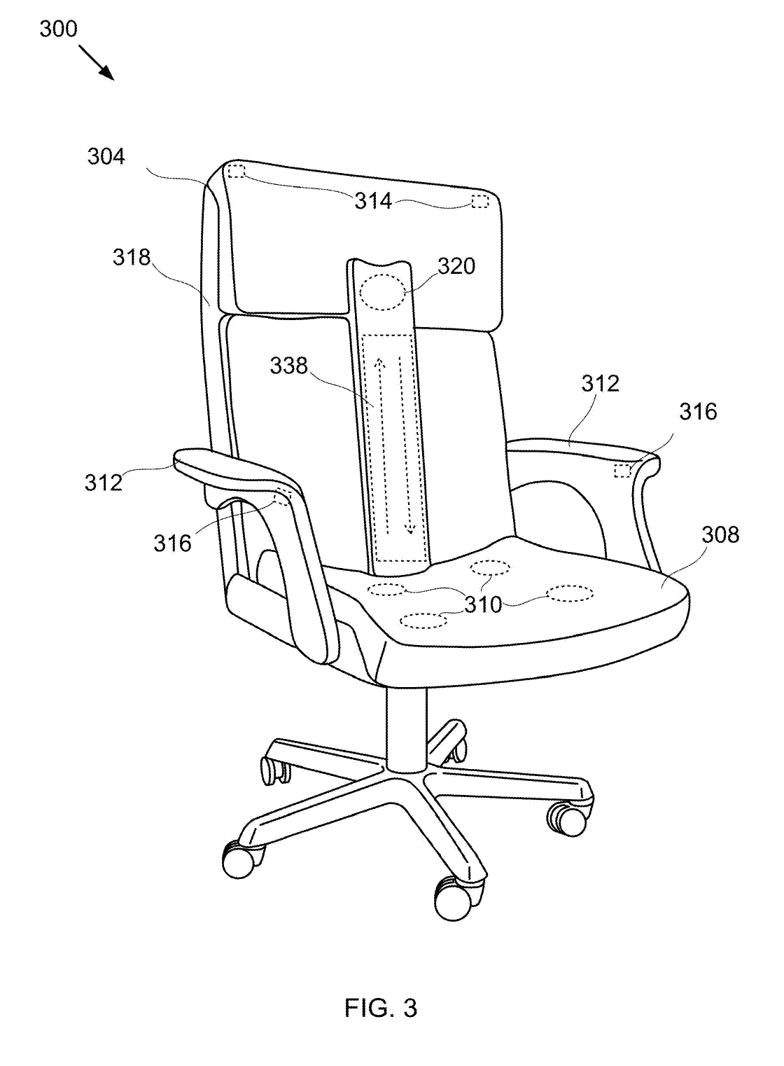

Referring to FIG. 3, an isometric view of a chair is shown at 300, as per one embodiment of the present invention. Chair 304 comprises sensors configured to detect at least one position of the human body and devices to produce physical reminders. This chair can be used with a controller, as in FIG. 1. Seat 308 of chair 304 comprises weight sensors 310, which detect the weight distribution of the human body sitting in chair 304. Arms 312 of chair 304 comprise position sensors 316. Position sensors 316 are placed in the fronts of arms 312 and their locations in three-dimensional space are detected by position receivers (not shown). Back 318 of chair 304 comprises pressure sensor 320, and sliding vibratory device 338. Pressure sensors 320 detect whether the user's shoulders are positioned correctly against the back of chair 304. Sliding vibratory device 338 provides a physical reminder of improper body positioning by causing vibrations to propagate up and down back 318 of chair 304.

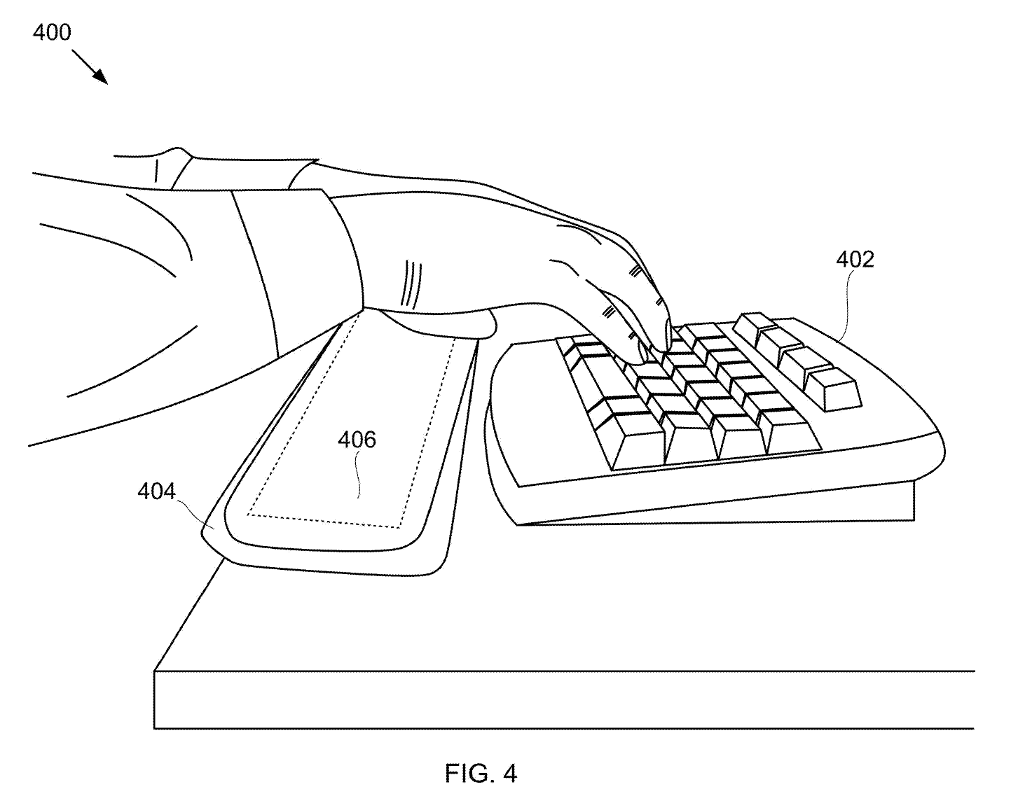

Referring to FIG. 4, an isometric view of a keyboard with wrist support is shown at 400, as per one embodiment of the present invention. Keyboard 402 is provided with wrist support 404. Wrist support 404 comprises pressure sensor 406. Pressure sensor 406 detects pressure from the user's wrist when the user types with incorrect wrist position.

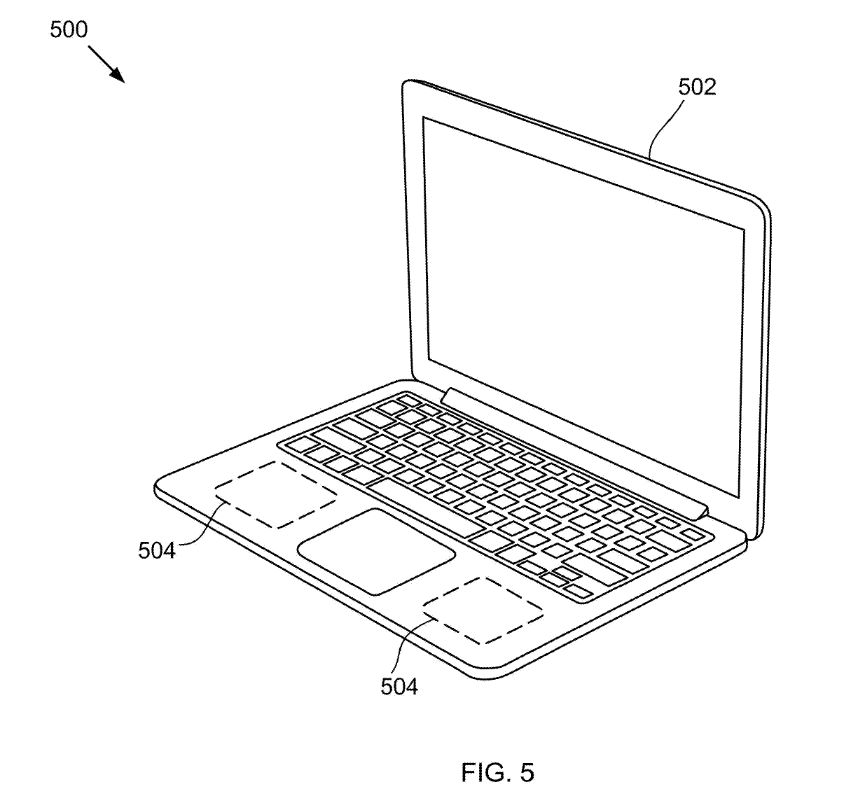

Referring to FIG. 5, an isometric view of a laptop is shown at 500, as per one embodiment of the present invention. Laptop 502 comprises pressure sensors 504. Pressure sensors 504 detects pressure from the user's wrist when the user types with incorrect wrist position.

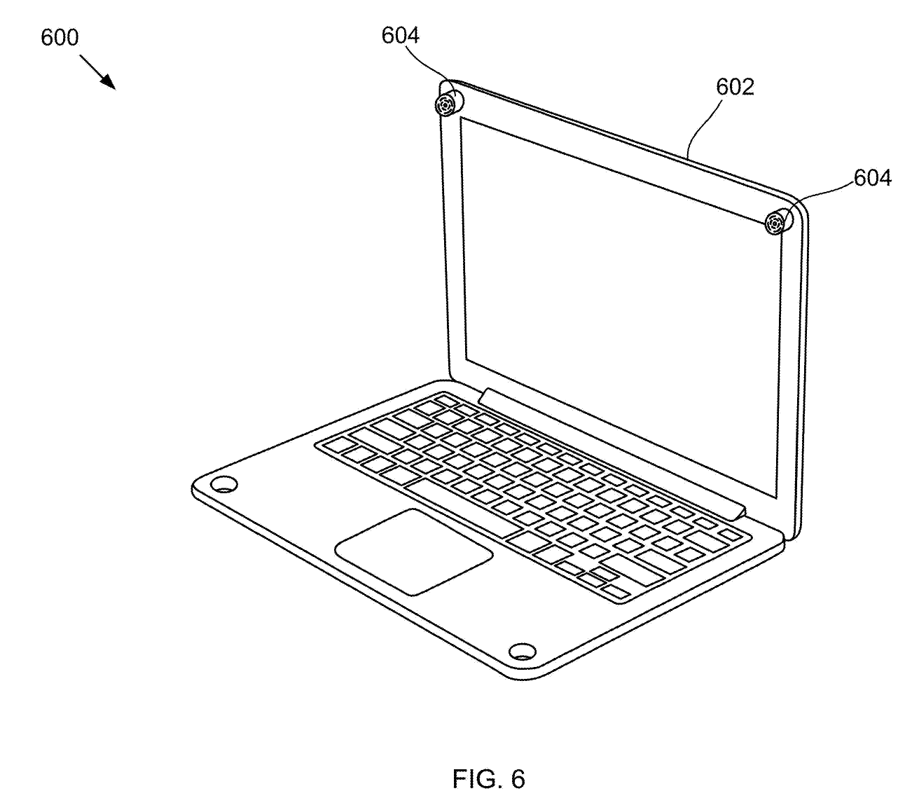

Referring to FIG. 6, an isometric view of a laptop is shown at 600, as per one embodiment of the present invention. Laptop 602 comprises ultrasonic transmitters/receivers 604, which produce ultrasonic sound which is reflected off the user. This ultrasonic sound is then detected by ultrasonic transmitters/receivers 604. The signals from ultrasonic transmitters/receivers 604 are evaluated in the controller to form a three-dimensional map of the user and determine deviations in body positioning.

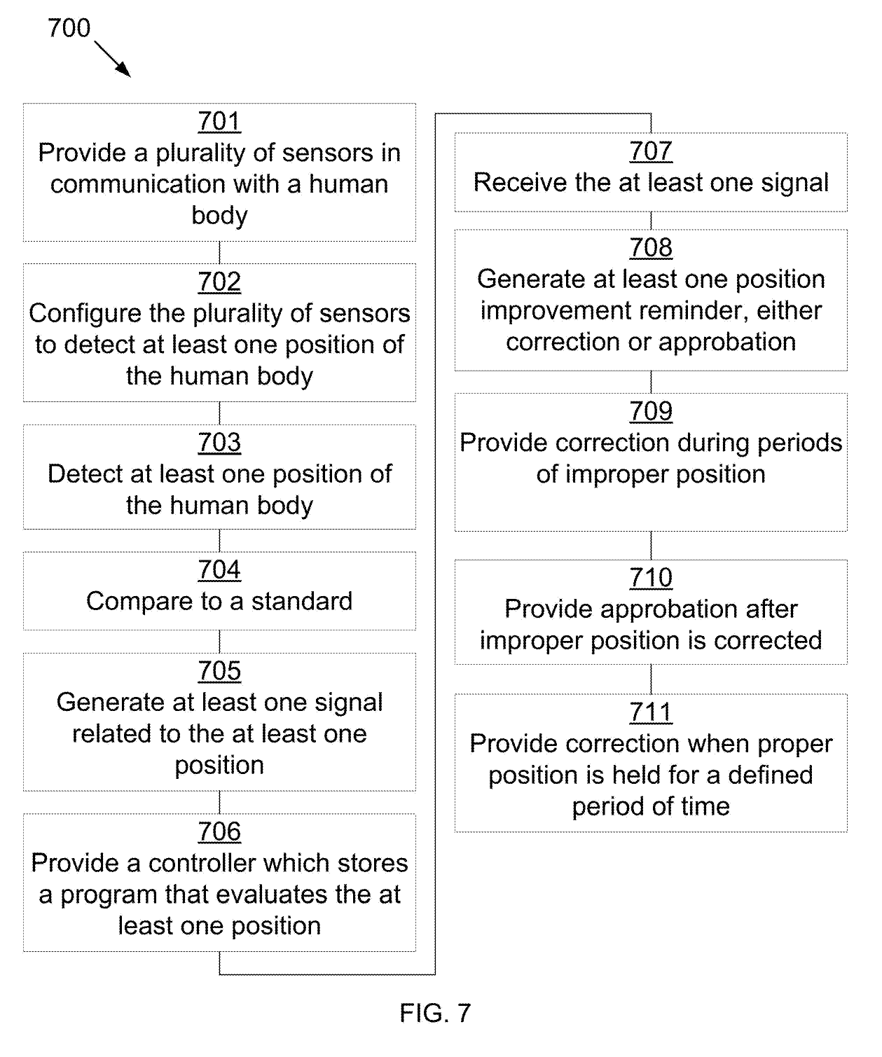

Referring to FIG. 7, a method for improving body positioning is shown at 700, as per one embodiment of the present invention. A plurality of sensors in communication with a human body are provided 701. The plurality of sensors comprises a plurality of weight sensors, one or more pressure sensors, one or more position sensors, a plurality of ultrasonic transmitters/receivers, or combinations thereof. Each of the plurality sensors is configured 702 to detect at least one position of the human body 703, compare the position to a standard 704, and to generate at least one signal related to the at least one position 705. A predetermined human body posture standard, or a user defined human body posture standard, may be visually displayed to a user as the sensors are activated. A controller is provided which stores a program that evaluates the at least one position 706, receives the at least one signal 707, and generates at least one position improvement reminder selected from the group comprising a visual reminder, an auditory reminder, and a physical reminder 708. The at least one position improvement reminder includes corrections and approbations, both selected from the group comprising visual, auditory, and physical notifications. The corrections continue as the improper position is detected 709. The approbations are provided after the improper position is corrected 710 and as positive feedback after intervals of proper position 711.

In some embodiments, the plurality of weight sensors are positioned in a seat of a chair such that a weight distribution of the human body sitting in the chair is evaluated.

In some embodiments, the one or more pressure sensors are positioned in or on a top-back portion of the chair; in or on a seat of the chair; in or on a floor; in or on a laptop such that wrists rest on the one or more pressure sensors when using an improper typing position; in or on a desk such that the wrists rest on the one or more pressure sensors when using the improper typing position; in or on a wrist support such that the wrists press on the one or more pressure sensors when using the improper typing position; on a wrist or wrists such that the wrists press the one or more pressure sensors on a desktop or laptop when using the improper typing position; or, in or on a combination thereof.

In some embodiments, the one or more position sensors are positioned in or on a mouse, keyboard, chair, monitors, ID badge, glasses, shoes, shirt, pants, hat, hair accessory, or a combination thereof. In some embodiments, one or more position receivers are provided that receive signals from the one or more position sensors, wherein the signals from the one or more position receivers are evaluated to triangulate a location in space of the one or more position sensors and the one or more position sensors comprise real-time locating system tags comprising radio frequency communication, infrared communication, acoustic communication, or combinations thereof.

In some embodiments, the plurality of ultrasonic transmitters/receivers produce ultrasonic sound and detect reflections of the ultrasonic sound around the human body and produce a signal for use in producing a three-dimensional map of the human body and evaluating the at least one position of the human body.

In some embodiments, the controller is a personal computer comprising a webcam, the webcam sensing the at least one position.

In some embodiments, the physical notifications further comprise vibrating a computer mouse, vibrating a keyboard, or combinations thereof.

In some embodiments, the visual notifications comprise on-screen text notifications, on-screen images, flashing lights, blinking lights, or combinations thereof. A predetermined human body posture standard, or a user defined human body posture standard, may be visually displayed to a user as the sensors are activated.

In some embodiments, the auditory notifications comprise computer beeps, audio or music notifications over computer speakers or headphones, or combinations thereof.

In some embodiments, the chair is powered by rechargeable batteries. In some embodiments, the chair is powered by induction coils in the floor.

1. A method for improving body positioning comprising:
providing a plurality of sensors in communication with a human body sitting in a chair at a table or desk or standing at a table or desk, the plurality of sensors comprising a plurality of weight sensors, one or more pressure sensors, one or more position sensors, a plurality of ultrasonic transmitters/receivers, or combinations thereof;
configuring each of the plurality of sensors to detect at least one position of the human body and to generate at least one signal related to the at least one position;
providing a controller to receive the at least one signal from each of the plurality of sensors, store a program that evaluates the at least one position from each of the plurality of sensors for any improper position, and, for each improper position, generates at least one position improvement reminder, wherein:
the at least one position improvement reminder includes corrections and approbations, wherein corrections comprise physical notifications, and approbations comprise visual, auditory, or visual and auditory notifications, the corrections continuing during a period the improper position is detected, the approbations being provided after the period and as positive feedback after intervals of proper position;
the physical notifications comprise vibrating a keyboard wrist support on the desk and vibrating a mouse wrist support on the desk.

2. The method of claim 1, positioning the plurality of weight sensors in a seat of the chair such that a weight distribution of the human body sitting in the chair is evaluated.

3. The method of claim 1, positioning the one or more pressure sensors:
in or on a top-back portion of the chair;
in or on a seat of the chair;
in or on a floor;
in or on a laptop such that wrists rest on the one or more pressure sensors when using an improper typing position;
in or on a desk such that the wrists rest on the one or more pressure sensors when using the improper typing position;
in or on a wrist support such that the wrists press on the one or more pressure sensors when using the improper typing position;
on a wrist or wrists such that the wrists press the one or more pressure sensors on a desktop or laptop when using the improper typing position; or,
in or on a combination thereof.

4. The method of claim 1, positioning the one or more position sensors in or on a mouse, keyboard, the chair, the monitors, ID badge, glasses, shoes, shirt, pants, hat, hair accessory, or a combination thereof.

5. The method of claim 1, further comprising providing one or more position receivers that receive signals from the one or more position sensors.

6. The method of claim 5, wherein the signals from the one or more position receivers are evaluated to triangulate a location in space of the one or more position sensors.

7. The method of claim 6, wherein the one or more position sensors comprise real-time locating system tags.

8. The method of claim 7, wherein the real-time locating system tags comprise radio frequency communication, infrared communication, acoustic communication, or combinations thereof.

9. The method of claim 1, producing ultrasonic sound and detecting the reflections of the ultrasonic sound around the human body with the plurality of ultrasonic transmitters/receivers, producing a signal for use in producing a three-dimensional map of the human body, and evaluating the at least one position of the human body.

10. The method of claim 1, wherein the corrections provided during the period the improper position is detected increase in frequency and amplitude as the period extends.

11. The method of claim 10, wherein the approbations provided after the period become more positive as the period decreases in duration.

12. The method of claim 1, wherein the approbations provided become more positive as the intervals of proper position increase in duration.

13. The method of claim 1, wherein the controller transmits the at least one signal from each of the plurality of sensors to a medical provider.

14. The method of claim 1, generating the physical notifications further comprising vibrating a computer mouse, vibrating a keyboard, vibrating portions of the chair, electrically shocking a lower back portion of the human body, or combinations thereof.

15. The method of claim 1, generating the visual notifications comprising on-screen text notifications, on-screen images, flashing lights, blinking lights, or combinations thereof.

16. The method of claim 1, generating the auditory notifications comprising computer beeps, audio or music notifications over computer speakers or headphones, or combinations thereof.

17. The system of claim 1, wherein the controller is a personal computer comprising a webcam, the webcam comprising a visual sensor.

18. A method for improving body positioning comprising:
providing a plurality of sensors in communication with a human body sitting in a chair at a table or desk or standing at a table or desk, the plurality of sensors comprising a plurality of weight sensors, one or more pressure sensors, one or more position sensors, one or more position receivers that receive signals from the one or more position sensors, and a plurality of ultrasonic transmitters/receivers;
configuring each of the plurality of sensors to detect at least one position of the human body and to generate at least one signal related to the at least one position;
positioning the plurality of weight sensors in a seat of the chair such that a weight distribution of the human body sitting in the chair is evaluated;
positioning the one or more pressure sensors in or on a top-back portion of the chair; in or on a seat of the chair; in or on a floor; in or on a laptop such that wrists rest on the one or more pressure sensors when using an improper typing position; in or on a desk such that the wrists rest on the one or more pressure sensors when using the improper typing position; in or on a wrist support such that the wrists press on the one or more pressure sensors when using the improper typing position; on a wrist or wrists such that the wrists press the one or more pressure sensors on a desktop or laptop when using the improper typing position; or, in or on a combination thereof;
positioning the one or more position sensors in or on a mouse, the keyboard, the chair, the monitors, ID badge, glasses, shoes, shirt, pants, hat, hair accessory, or a combination thereof;
evaluating the signals from the one or more position receivers to triangulate a location in space of the one or more position sensors, the one or more position sensors comprising real-time locating system tags comprising radio frequency communication, infrared communication, acoustic communication, or combinations thereof;
producing ultrasonic sound and detecting the reflections of the ultrasonic sound around the human body with the plurality of ultrasonic transmitters/receivers, producing a signal for use in producing a three-dimensional map of the human body, and evaluating the at least one position of the human body; and,
providing a controller to receive the at least one signal from each of the plurality of sensors, store a program that evaluates the at least one position from each of the plurality of sensors for any improper position, and, for each improper position, generates at least one position improvement reminder, wherein:
the at least one position improvement reminder includes corrections and approbations, wherein corrections comprise physical notifications, and approbations comprise visual, auditory, or visual and auditory notifications, the corrections continuing during a period the improper position is detected, the approbations being provided after the period and as positive feedback after intervals of proper position;
the physical notifications comprise vibrating a keyboard wrist support on the desk and vibrating a mouse wrist support on the desk, vibrating the mouse and vibrating the keyboard, or combinations thereof;
the visual notifications comprise on-screen text notifications, on-screen images, flashing lights, blinking lights, or combinations thereof; and,
the auditory notifications comprise computer beeps, audio or music notifications over computer speakers or headphones, or combinations thereof.

19. The method of claim 18, wherein the corrections provided during the period the improper position is detected increase in frequency and amplitude as the period extends, the approbations provided after the period become more positive as the period decreases in duration, the approbations provided become more positive as the intervals of proper position increase in duration, and the controller transmits the at least one signal from each of the plurality of sensors to a medical provider.

20. The method of claim 1, wherein a predetermined human body posture standard, or a user defined human body posture standard, is visually displayed to a user as the sensors are activated.
